(19) United States
(12) Patent Application Publication
(10) Pub. No.: US 2011/0250274 A1
(43) Pub. Date: Oct. 13, 2011

(54) ESTRIOL FORMULATIONS

(76) Inventors: Ze'ev Shaked, San Antonio, TX (US); Walter Elger, Berlin (DE); Klaus Nickisch, Berlin (DE)

(21) Appl. No.: 13/063,803

(22) PCT Filed: Sep. 18, 2009
(86) PCT No.: PCT/US2009/057524

§ 371 Date: Jun. 6, 2011

Related U.S. Application Data
(60) Provisional application No. 61/098,615, filed on Sep. 19, 2008.

(51) Int. Cl. A61K 31/565; A61P 5/30; A61K 9/00

(52) U.S. Cl. 424/484; 514/182; 514/171

(57) ABSTRACT

Disclosed herein are oral dosage forms and methods of their use, in particular oral dosage systems for the delivery of estriol compounds. Embodiments described herein relate to rapidly disintegrating oral dosage formulations that disintegrate in the saliva of the buccal and/or sublingual and/or esophageal cavity. Oral dosage forms described herein relate to stabilized amorphous and nanocrystalline forms of the active ingredients of the formulations.

### BACKGROUND OF THE INVENTION

1. Field of the Invention

The invention generally relates to oral dosage forms and methods of their use, in particular oral dosage systems for the delivery of drugs for the treatment of perimenopause conditions. More particularly, this invention generally relates to methods to orally administer dosage forms of estriol compounds.

2. Description of the Relevant Art

Tablets or films that disintegrate or dissolve rapidly in a patient's mouth without the use of the water are convenient for patients that have difficulty swallowing oral dosage forms and situations where certain compliance conditions are needed. For these rapidly dissolving oral forms, a small amount of saliva is sufficient to disintegrate or dissolve such forms in the oral cavity. The drug released from these tablets/films can be absorbed partially or completely into the systemic circulation from the buccal or sublingual cavity, or can be swallowed as a solution to be absorbed through the gastrointestinal tract. The sublingual route usually produces a faster onset of action than traditionally administered tablets and the portion absorbed through the sublingual mucosa bypasses the hepatic first pass metabolic processes. In addition, the sublingual dosage forms do not get exposed to the acidic environment of the stomach. The decreased production of endogenous female sex hormones during perimenopause induces various physical and psychological symptoms that can result in side effects, ranging from annoying to life-altering, that impact the overall quality of life. Hormone replacement therapy (HRT) is considered to be effective for perimenopausal symptoms such as hot flushes and night sweats, and is helpful in the prevention of osteoporosis and atrophic changes in the genital tract. The effect of HRT is dependent on the prolonged and continuous use of estrogens for many years. Estriol [estra-1,3,5(10)-triene-3,16a,17β-triol], a natural estrogenic hormone, is one of the predominant steroid hormones found in women. Estriol is the preferred hormone for use in HRT, because it is the only hormone among the three main human estrogens that has no mitogenic activity. Estriol has been shown to protect the reproductive organs by means of its receptor competition with the endogenous estrogens. Further, estriol is one of the most abundant of the natural estrogens in the blood stream. Since estriol has considerably weaker stimulatory effects on endometrial proliferation than estradiol, it is associated less frequently with genital bleeding and may not require concomitant use of progestin. When administered orally in the form of tablets and capsules, estriol is absorbed and eliminated rapidly compared to other estrogens. Almost 90% of the administered oral dose is rapidly glucuro-conjugated by the liver. As a result, only 1-2% of the oral dose administered reaches the systemic circulation. For this reason, for oral administration of estriol to be effective, a dose of 2-8 mg per day has been found to be necessary to prevent vaginal atrophy and to be effective in the treatment of hot flashes, disturbed sleep and several other problems associated with the perimenopause state. Because of the relatively high dose (2-8 mg/day) administered, there is a possibility of side effects occurring such as nausea and mastalgia. Furthermore, this massive inactivation of orally administered estriol is influenced by the time of administration and food intake. This variability in the circulating levels of orally administered estriol makes it difficult to tailor the dosage to the optimal range. This “first pass” effect may also lead to an undesirable increase in the production of certain coagulation factors and other biologically important compounds by the liver. Parenteral administration of estriol avoids the aforementioned problems associated with oral administration, but such an invasive mode of administration is generally undesirable. Transdermally administered estriol is also suitable for the therapy of perimenopausal induced osteoporosis. However, the very high estriol amounts (12 mg per 24 hours) which had to be continuously administered transdermally in order to achieve a serum level of free estriol that corresponded to the physiological concentrations of estrogenic hormones in the female cycle (50 to 350 μg/ml) must be considered to be disadvantageous. Apart from this, the transdermal administration of estriol should result in a largely constant blood level of estriol which has a positive effect on the bones, but also promotes the unwanted side effects on the mucosa of the uterus. Estriol has also been administered by a vaginal route through the use of creams, pessaries, depot suppositories, vaginal rings, etc. It was found that intravaginal estriol is rapidly absorbed and is suitable for local and systemic estrogen replacement therapy, and that it was more effective than the oral regimen. By avoiding enterohepatic circulation, high plasma levels of free estriol (20%) may be obtained and produces the expected pharmacodynamic effect. Following the intravaginal administration of 0.5 mg estriol, systemic levels higher than those obtained with 8 mg of oral administered estriol were obtained. Given the difficulty in administering the vaginal formulation, it is not a preferred route by patients. Oral estriol would be the preferred route, however, orally administered estriol is significantly inactivated by first-pass metabolism. Additionally, oral absorption of estriol is also modified by the time of administration or by food, and more stable circulating levels are difficult to achieve. Given the difficulties associated with both routes of administration, there exists a need for a convenient dosage form that can provide easy oral administration of estriol, that can provide significantly higher drug levels, and its performance is not modified by the time of administration or presence of food. Furthermore by achieving higher estriol levels at low drug exposure, there will be a marked improvement in postmenopausal complaints associated with stimulation of the endometrial proliferation. The lack of endometrial stimulus will eliminate the need to restore artificial menses with progesterone administration and thus avoid one of the major causes of discomfort and withdrawal from estrogen replacement therapy.

### SUMMARY OF THE INVENTION

The embodiments described herein relate oral dosage formulations that disintegrate or disperse in the saliva of the oral cavity to release an estriol compound. Additional embodiments relate to compositions for nasal administration of estriol compounds. These methods avoid hepatic first-pass by allowing for absorption of the estriol compounds through the oral, buccal, and sublingual mucosa or, in the case of nasal inhalants, the nasal mucosa. In certain embodiments, oral dosage forms for use in the treatment of perimenopausal symptoms include an estriol compound and a pharmaceutically acceptable matrix material, wherein the oral dosage form releases at least about 90% of the estriol compound in a time of less than about 300 seconds when contacted with saliva of the buccal cavity. In one embodiment, the oral dosage form may be in a tablet form. The oral dosage form may include between about 0.01 mg to about 2 mg of the estriol compound. At least a portion of the estriol compound may be in a stabilized amorphous form and/or nanocrystalline form. In other embodiments, at least a portion of the estriol compound may be in a micronized form. Suitable pharmaceutically acceptable matrix materials for the oral dosage form include, but are not limited to: polyethylene glycol polymers, cellulose based matrix materials, a vinyl based matrix material, a polyvinyl pyrrolidone polymer, an acrylic acid-based polymer, a methacrylic acid-based polymer, an acrylic acid—methacrylic acid based copolymer, or a mixture thereof. In some embodiments, the oral dosage form may include one or more poloxamers. In some embodiments, the oral dosage form may include one or more surfactants. In certain embodiments, a method of treating perimenopausal conditions in a subject includes administering to a subject an oral dosage form including an effective amount of an estriol compound and a pharmaceutically acceptable matrix material, wherein the oral dosage form releases at least about 90% of the estriol compound in a time of less than about 300 seconds when contacted with saliva of the buccal and/or sublingual cavity of the subject. In certain embodiments, an oral dosage form is produced by melting an estriol compound with one or more hydrophilic polymers. In one embodiment, the resulting molten dispersion may be atomized in a suitable fluid-bed processor using a spray congealing process. Melting of the estriol compound with one or more hydrophilic polymers may be accomplished in a heated high-shear mixer. In another embodiment, the molten dispersion may be produced and/or extruded using a melt-extrusion process. In some embodiments, an oral dosage form is produced by melting an estriol compound with one or more hydrophilic polymers in the presence of a chemically and/or physically inert filler material using a melt extruder. In certain embodiments, an oral dosage an oral dosage form is produced by dissolving or dispersing an estriol compound in a solvent, a mixture solvents, or a low melting material (e.g. wax), with one or more hydrophilic polymers, followed by mixing the resulting solution or dispersion with a chemically and/or physically inert filler material using a melt extruder. In certain embodiments, an oral dosage form as described above, is produced by forming a mixture of an estriol compound in a supercritical fluid; removing the supercritical fluid from the estriol compound; combining the supercritical fluid treated estriol compound with a pharmaceutically acceptable matrix material; and forming an oral dosage form from the combined supercritical fluid treated estriol compound and pharmaceutically acceptable matrix material, wherein the formed oral dosage form releases at least about 90% of the estriol compound in a time of less than about 300 seconds when contacted with saliva of the buccal and/or sublingual cavity. In some embodiments the supercritical fluid is carbon dioxide. Other supercritical fluids known in the art may be used. In some embodiments, the mixture of estriol compound in a supercritical fluid includes one or more poloxamers and/or one or more surfactants. In certain embodiments, an oral dosage form as described above, is formed by forming a mixture of an estriol compound and a pharmaceutically acceptable matrix material; forming an estriol solid dispersion from the mixture; milling the mixture to form particles of the estriol solid dispersion; and forming an oral dosage form from the estriol solid dispersion particles and a pharmaceutically acceptable matrix material, wherein the formed oral dosage form releases at least about 90% of the estriol compound in a time of less than about 300 seconds when contacted with saliva of the buccal and/or sublingual cavity.

### DETAILED DESCRIPTION OF THE PREFERRED EMBODIMENTS

Embodiments described herein relate to oral dosage forms that are designed to reduce or inhibit symptoms associated with perimenopause. Furthermore, the embodiments described herein are directed to methods of formulating such oral dosage forms. Additionally, embodiments described herein provide methods of administering such oral dosage forms. Mucous membranes such as the mucosa of the buccal and sublingual cavity have several physical attributes, such as a rich blood supply, that makes them desirable sites for administration of active agents for systemic delivery. Transmucosal delivery of active agents further avoids first-pass metabolism by the liver as well as poor uptake or inactivation via the gastrointestinal pathway. In one embodiment, estriol compounds may be formulated into a fast disintegrating buccal or sublingual tablets (e.g. oral disintegrating tablets or films) that includes a suitable dose that would result in plasma estriol levels similar to those of produced by the recommended vaginal dose (approx. 0.5 mg of estriol for adults), by selecting the appropriate form of the drug (e.g., an amorphous form, a nanocrystalline form or mixture). In other embodiments, estriol compounds may be delivered as a nasal/pulmonary inhalant. Estriol [estra-1,3,5(10)-triene-3,16a,17β-triol], a natural estrogenic hormone, and is one of the predominant steroid hormone found in women. Estriol derivatives that may be used in hormone replacement therapy include suitable pharmacologically acceptable esters of estriol including, but not limited to, estriol triacetate, estriol tripropionate, estriol-3-acetate, estriol-16-acetate, estriol-16,17-diacetate, estriol-3-17-disulfate, estrio1-16,17-disulfate, estriol-3-sulfate, estrio1-17-sulfate, estriol-3-hemisuccinate or estriol-16,17-hemisuccinate. Further estriol derivatives include glycoester derivatives and other derivatives as described in U.S. Pat. Nos.: 4,952,569; 4,780,460; and 4,738,957, each of which is incorporated herein by reference. Pharmaceutically acceptable salts of estriol and estriol derivatives may also be used in any of the embodiments described herein. As used herein the term “estriol compounds” refers to the compound estriol, estriol derivatives, and pharmaceutically acceptable salts of estriol and estriol derivatives. In some embodiments the oral dosage form is monolithic and substantially solid, that is, it is formed as a unitary mass that is molded, freeze dried, cut, ground or otherwise formed in its final shape. In other embodiments, the oral dosage form may be an aggregate or composite of individual solid particulates, pellets, beads, granules, sprinkles, triturates, microspheres or the like formed into a tablet or disposed in a capsule. The phrase “oral dosage form” as used herein refers to pharmaceutical compositions formed as tablets, caplets, softlets, films, troche, sachet, wafers and the like. In some embodiments, the oral dosage forms are capable of disintegrating or dissolving when contacted with saliva of the buccal and/or sublingual cavity. In one embodiment, oral dosage forms described herein used to reduce or inhibit at least some symptoms associated with perimenopause include an estriol compound in amounts effective to reduce or inhibit at least some perimenopausal symptoms. An oral dosage form may include, but is not limited to, between about 0.01 mg to about 2.0 mg, between about 0.02 mg to 1.5 mg, or between about 0.05 mg to 1.0 mg of an estriol compound. An oral dosage form includes, but is not limited to, 0.01 mg, 0.02 mg, 0.05 mg, 0.1 mg, 0.25 mg, 0.5 mg, 0.75 mg, 1 mg, or 1.5 mg of an estriol compound. While the oral dosage forms described herein are generally directed to estriol compounds, it should be understood that the oral dosage forms may be modified for other combinations of active ingredients. For example, the oral dosage forms described herein may include estriol compounds in combination with one or more progestogens such as norethindrone, norethindrone acetate, drospirenone, levonorgestrel, desogestrel, gestodene, norgestrel, ethynodiol diacetate, norgestimate, cyproterone acetate, or norgestimate. In some embodiments, the estriol compound may be in a stabilized amorphous and/or nanocrystalline form to improve the solubility of the estriol compound in aqueous solutions (e.g., saliva) and, therefore, improve their bioavailability. Estriol, and many estriol compounds, are typically obtained in a crystalline form. As used herein the term “crystalline” refers to solids in which the atoms are arranged in fixed geometric patterns or lattices. Crystalline forms of estriol compounds tend to be less soluble in aqueous environments due to their ordered structure, which requires a higher amount of energy to dissolve. Use of stabilized amorphous and or nanocrystalline forms of estriol compounds tends to improve the solubility of these compounds As used herein the term “stabilized amorphous” refers to solids in which the atoms are arranged in a random order. Amorphous solids tend to be easier to dissolve since the additional stability gained by crystalline compounds is lost when the atoms are disordered. Another way to increase the solubility of the estriol compound is through the formation of nanocrystals. As used herein the term “stabilized nanocrystalline” refers to crystals having an average particle size of less than about 1 μm and which have been formed without grinding or milling of the particles. Specifically, stabilized nanocrystalline solids are formed by a precipitation process which produces particles having an average dimension of less than 1 μm. Stabilized nanocrystalline particles do not include particles formed by reducing the average particle size (e.g., milling, grinding, etc.) of a crystalline form of the solid to dimensions that are less than 1 μm. Amorphous and non-crystalline forms of estriol compounds may be formed using a number of techniques. Examples of such techniques include dissolution/evaporation, dissolution/precipitation and supercritical fluid techniques. In a dissolution/evaporation process, the estriol compound is dissolved in a solvent, or a mixture of solvents, and the estriol compounds are recovered by evaporating the solvent. Evaporation of the solvent causes the initial morphology of at least a portion of the estriol compounds to be changed to an amorphous and/or microcrystalline form. In a dissolution/precipitation process, the estriol compound is dissolved in a first solvent, or a first mixture of solvents. A second solvent, or a second mixture of solvents, is added to the estriol solution to cause at least a portion of the estriol compounds to precipitate out in an amorphous and/or microcrystalline form. In another embodiment, estriol compounds are contacted with a supercritical fluid to convert at least a portion of the estriol compound into an amorphous state and/or nanocrystalline state. Many different supercritical fluids may be used including, but not limited to, supercritical fluids of carbon dioxide, n-butane, dimethyl ether, methane, ethane, propane, ethylene, propylene, methanol, ethanol, acetone and xenon. These supercritical fluids are highly tunable solvents in which small changes in pressure and temperature may lead to changes in fluid density. The change in fluid density may lead to changes in the solvent properties of the supercritical fluid. Thus by modifying the temperature and/or pressure, of the supercritical fluid, the fluid density of the supercritical fluid is altered which, in turn, alters the solubility properties of the solutes dissolved therein. Altering the fluid density allows the morphology of the particles produced by the supercritical fluid process to be changed. In some embodiments, altering the temperature and/or pressure of the supercritical fluid allows the morphology of the solutes to be changed from crystalline to amorphous. In one embodiment, super critical carbon dioxide is used to alter the estriol compound from the crystalline to the amorphous/nanocrystalline states. Additionally, supercritical carbon dioxide has low toxicity, is non-flammable and environmentally compatible. It is easily removed from the system once processing is completed. One supercritical fluid technique that may be used is a process known as Solution Enhanced Dispersion by Supercritical Fluids. Details regarding the apparatus and process may be found in U.S. Pat. No. 5,851,453 to Hanna et al. which is incorporated herein by reference. In one embodiment, an estriol composition may include a solid dispersion of one or more estriol compounds in a pharmaceutically acceptable matrix composition. A pharmaceutically acceptable matrix composition may include one or more pharmaceutically acceptable matrix materials and, optionally, one or more excipients. As used herein the term “solid dispersion”, means that a compound is substantially evenly distributed through the polymer, either as a solid suspension in the polymer or dissolved within the polymer matrix. The estriol composition may be used to form a tablet, a film or a composition suitable for nasal administration, for the treatment of perimenopausal symptoms. Pharmaceutically acceptable matrix materials are those materials indicated to be generally regarded as safe (“GRAS-certified”) or national formulary certified. As used herein, a polymer is considered hydrophilic, water-soluble, or water swellable if it is more than sparingly soluble as defined by USP 29/NF 24, that is if according to USP 29/NF 24 the polymer is classified as “soluble” or “very soluble.” As used herein, a polymer is considered to be hydrophobic or water-insoluble if it is “sparingly soluble” or “practically insoluble” or “insoluble” as defined by USP 29/NF 24. Pharmaceutically acceptable matrix materials may include cellulose based matrix materials, vinyl based matrix materials, acrylic acid—methacrylic acid based matrix materials and other synthetic and natural materials. Cellulose based matrix materials include, but are not limited to: ionic and nonionic cellulose compounds. Examples of ionic cellulose compounds include, but are not limited to, carboxymethylcellulose (CMC), sodium carboxymethylcellulose, calcium carboxymethylcellulose, carboxyethylcellulose (CEC), hydroxyethylmethylcellulose acetate phthalate, hydroxyethylmethylcellulose acetate succinate, hydroxypropylmethylcellulose phthalate (HPMCP), hydroxypropylmethylcellulose succinate, hydroxypropylcellulose acetate phthalate (HPCAP), hydroxypropylcellulose acetate succinate (HPCAS), hydroxypropylmethylcellulose acetate phthalate (HPMCAP), hydroxypropylmethylcellulose acetate succinate (HPMCAS), hydroxypropylmethylcellulose acetate trimellitate (HPMCAT), hydroxypropylcellulose butyrate phthalate, cellulose acetate phthalate (CAP), cellulose acetate butyrate, cellulose acetate succinate, cellulose acetate trimellitate (CAT), cellulose acetate terephthalate, cellulose acetate isophthalate, cellulose propionate phthalate, cellulose propionate trimellitate, cellulose butyrate trimellitate, methylcellulose acetate phthalate, methylcellulose phthalate, and ethylhydroxycellulose phthalate. Exemplary nonionic cellulose derivatives include cellulose, microcrystalline cellulose, alkyl celluloses, and hydroxyalkyl celluloses. Examples of alkyl celluloses include, but are not limited to methylcellulose (MC), ethylcellulose (EC), and propylcellulose. Examples of hydroxyalkyl celluloses include, but are not limited to, hydroxyethylcellulose (HEC), hydroxypropylcellulose (HPC), hydroxymethylcellulose hydroxypropylmethylcellulose (HPMC), hydroxypropylmethylcellulose acetate, hydroxyethylmethylcellulose, hydroxyethylcellulose acetate, amd hydroxyethylethylcellulose. Vinyl based matrix matrerials include, but are not limited to, polyvinyl acetate phthalate (PVAP), polyvinylbutyrate acetate, polyvinyl acetate, vinyl acetate-maleic anhydride copolymer, and polyvinyl acetal diethylaminoacetate. Acrylic acid-based polymers, methacrylic acid based polymers, and acrylic acid—methacrylic acid based copolymers may also be used as a pharmaceutically acceptable matrix material. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid. Examples of acrylic acid-based polymers include carbomers (e.g., Carbopol® polymers, Lubrizol Advanced Materials, Inc.). Examples of pharmaceutically acceptable acrylic acid based Carbopol® polymers include Carbopol polymers composed of polymers of acrylic acid crosslinked with allyl sucrose or allyl pentaerythritol. Examples of useful Carbopol® polymers includes, but are not limited to, Carbopol® 71G NT, Carbopol® 971P NF, Carbopol® 974P NF, Carbopol® 980 NF, Carbopol® 981 NF, Carbopol® 5984 EP, Carbopol® Ultrez 10 NF, Carbopol® 934 NF, Carbopol® 934P NF, Carbopol® 940 NF, Carbopol® 941 NF, and Carbopol® 1342 NF. Another class of acrylic acid-based polymers include polycarbophil polymers. Polycarbophil polymers are acrylilc acid polymers crosslinked with divinyl glycol. Examples of polycarbophil polymers include Noveon®0 AA-1 Polycarbophil Acid, Noveon® CA-1 Polycarbophil Calcium neutralized/Coarsely ground, and Noveon® CA-2 Polycarbophil Calcium neutralized/Finely ground (Lubrizol Advanced Materials, Inc.). As used herein, the phrase “acrylic acid—methacrylic acid based copolymers” refers to any polymer that includes one or more repeating units that include and/or are derived from acrylic acid and one or more repeating units that include and/or are derived from methacrylic acid. Examples of acrylic acid-methacrylic acid based copolymers include, but are not limited to, Eudragit® L 100-55, Eudragit® L 30 D-55, Eudragit® L 100, Eudragit® S 100, Eudragit® FS 30 D, Eudragit® RL 30 D, Eudragit® RL PO, Eudragit® RL 100, Eudragit ® RS 30 D, Eudragit® RS PO, Eudragit® RS 100, Eudragit® NE 30 D, Eudragit® NM 30 D, Eudragit® NE 40 D, Eudragit® E 100, Eudragit® E PO. Polyvinyl pyrrolidone (PVP) polymers may also be used as a matrix material or in combination with other matrix materials. The term “polyvinylpyrrolidone” or “PVP” refers to a polymer, either a homopolymer or copolymer, containing vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/ dimetlaylamino-ethylmethacrylate. The cross-linked homopolymer is insoluble and is generally known in the pharmaceutical industry under the designations polyvinylpolypyrrolidone, crospovidone and PVP. The copolymer vinylpyrrolidone-vinyl acetate is generally known in the pharmaceutical industry under the designations Copolyvidon(e), Copolyvidonum or VP-VAc. The term “soluble” when used with reference to PVP means that the polymer is soluble in water and generally is not substantially cross-linked, and has a molecular weight of less than about 2,000,000. Soluble PVP polymers have been identified under in the pharmaceutical industry under a variety of names, the most commonly used include Povidone, Polyvidon(e), Polyvidonum, Polyvidonum, poly (N-vinyl-2-pyrrolidinone, poly (N-vinylbutyrolactam), poly (1-vinyl-2-pyrrolidone), poly [1-(2-oxo-pyrrolidinyl) ethylene]. Monoalkyl esters of poly (methyl vinyl ether/maleic acid) may also be used as a pharmaceutically acceptable matrix material. Examples of Monoalkyl esters of poly (methyl vinyl ether/maleic acid) include Gantriz® polymers (International Specialty Products, Inc.). Examples of useful Gantriz® polymers includes, but are not limited to, Gantrez® ES-225, Gantrez® ES-425, Gantrez® MS-955, Gantrez® S-96 BF Solution ST, Gantrez® S-97 BF, and Gantrez® S-97 BF Solution. Further examples of pharmaceutically-acceptable matrix materials include, but are not limited to polymers based on derivatives of acrylic acid and methacrylic acid including, but not limited to, alkyl ester derivatives, alkylether ester derivatives, amide derivatives, alkyl amine derivatives, anhydride derivatives, cyanoalkyl derivatives, aminoalkyl methacrylate copolymers, carboxylic acid functionalized polymethacrylates, amine-functionalized polymethacrylates, and amino-acid derivatives. Other matrix materials that may be used include, but are not limited to, polyethylene oxide (PEO), polyethylene glycol (PEG), polypropyleneglycol, ethylene oxide-propylene oxide co-polymers, chitosan, polyvinyl alcohol (PVA), polyacrylamides, polyesters, polymethacrylamides, polyphosphazines, polyoxazolidines, polyhydroxyalkylcarboxylic acids, poly (vinyl acetal) diethylaminoacetate, polyvinyl alcohol/polyvinyl acetate (PVA/PVAc) copolymers, alginic acid and its derivatives such as carrageenate alginates, ammonium alginate, propylene glycol alginate, and sodium alginate, starch and starch derivatives, polysaccharides, carboxypolymethylene, natural gums such as gum guar, gum acacia, gum tragacanth, karaya gum and gum xanthan, povidone, gelatin, waxes, shellac, and zein. In addition to one or more pharmaceutically acceptable matrix material and one or more estriol compounds, an estriol composition may also include one or more functional excipients such as lubricants, fillers, antioxidants, buffering agents, alkalinizing agents, acidifying agents, disintegrants, diluents, sweeteners, chelating agents, colorants, flavorants, surfactants, solubilizers, wetting agents, stabilizers, enhancers, bioadhering/mucus retaining agents, preservatives, absorbents, cross-linking agents, bioadhesive polymers, retardants, pore formers, osmotic agents crystallization inhibitors, poloxamers, and fragrance. Tablet lubricants and glidants useful as an excipient include, but are not limited to magnesium stearate, sodium stearate, stearic acid (stearin), hydrogenated oil, waxes, colloidal silicon dioxide, micronized polyoxyethylene glycol, sodium stearyl fumarate and combinations thereof. Fillers useful as an excipient include, but are not limited to, lactose, starch, dextrose, sucrose, fructose, maltose, mannitol, sorbitol, microcrystalline cellulose, powdered cellulose or any combination of the foregoing. In an embodiment, the filler is a mixture of water-soluble fillers to reduce the chance of unpleasant grittiness when the tablet dissolves in the oral cavity of the patient. The filler may be a direct compression sugar such as confectioners sugar, dextrates, dextrin, dextrose, fructose, maltose, glucose, mannitol, polydextrose, sorbitol, xylitol, erythritol, or other sugars and sugar derivatives both in their crystalline, amorphous forms or mixture of both. As used herein, the term “antioxidant” is intended to mean an agent that inhibits oxidation and thus is used to prevent the deterioration of preparations by oxidation due to the presence of oxygen free radicals or free metals in the composition. Such compounds include, by way of example and without limitation, ascorbic acid (Vitamin C), ascorbyl palmitate, butylated hydroxyanisole (BHA), butylated hydroxytoluene (BHT), hypophophorous acid, monothioglycerol, sodium ascorbate, sodium formaldehyde sulfoxylate, sodium metabisulfite, sodium bisulfate, vitamin E and its derivatives, derivatives of selenium (selenomethionine), propyl gallate and others known to those of ordinary skill in the art. A buffering agent is used to resist change in pH upon dilution or addition of acid or alkali. Such compounds include, by way of example and without limitation, potassium metaphosphate, potassium phosphate, monobasic sodium acetate and sodium citrate anhydrous and dihydrate, salts of inorganic or organic acids, salts of inorganic or organic bases, and others known to those of ordinary skill in the art. As used herein, the term “alkalizing agent” is intended to mean a compound used to provide alkaline medium for product stability or for interaction with acidic compounds to produce effervescence of the composition. Such compounds include gas-releasing alkaline compounds and other alkaline compounds. As used herein, a gas-releasing alkaline compound is an alkaline compound that releases a gas, or causes a solution to effervesce, when exposed to a proton source such as an acidic agent or water. Examples of gas-releasing alkaline compounds include, but are not limited to, carbonate, bicarbonate, sesquicarbonate and hydrogencarbonate salts (hereinafter generically referred to as “carbonate salts”) of potassium, lithium, sodium, calcium, magnesium, and ammonium; L-lysine carbonate; arginine carbonate; sodium glycine carbonate, sodium amino acid carbonate; anhydrous sodium perborate; effervescent perborate; sodium perborate monohydrate; sodium percarbonate; and sodium dichloroisocyannurate. Other examples of alkalizing agents include diethanolamine, monoethanolamine, potassium hydroxide, sodium borate, sodium hydroxide, triethanolamine, sodium dihydrogen phosphate, arginine, lysine and meglumine. As used herein, the term “acidifying agent” is intended to mean a compound used to provide alkaline medium for product stability or for interaction with basic compounds to produce effervescence of the composition. Such compounds include, by way of example and without limitation, tartaric acid, citric acid, maleic acid, fumaric acid, malic acid, adipic acid, succinic acid, lactic acid, glycolic acid, alpha hydroxy acids, ascorbic acid, amino acids and their alkali hydrogen acid salts and others known to those of ordinary skill in the art. Further alkalizing agents and acidifying agents are described in Handbook of Pharmaceutical Excipients and U.S. Pat. No. 6,316,029, which are incorporated herein by reference. As used herein, the term “disintegrant” is intended to mean a compound used in solid dosage forms to promote the disruption of a solid mass (layer) into smaller particles that are more readily dispersed or dissolved. Exemplary disintegrants include, by way of example and without limitation, starches such as corn starch, potato starch, pre-gelatinized and modified starches thereof, sweeteners, microcrystalline cellulose (e.g., Avicel™), carboxymethylcellulose calcium, croscarmellose sodium, carmellose calcium, alginic acid, sodium alginate, cellulose polyacrilin potassium (e.g., Amberlite™), alginates, sodium starch glycolate, gums, agar, guar, locust bean, karaya, pectin, tragacanth, crospovidone and other materials known to one of ordinary skill in the art. A superdisintegrant is a rapidly acting disintegrant. Exemplary superdisintegrants include crospovidone (Polyplasdone) and low substituted HPC. Exemplary chelating agents include EDTA, EGTA, alpha-hydroxy fatty acids, polyamines, derivatives thereof, and others known to those of ordinary skill in the art. As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other FD&C dyes and natural coloring agents such as grape skin extract, beet red powder, beta carotene, annato, carmine, turmeric, paprika, and other materials known to one of ordinary skill in the art. The amount of coloring agent used will vary as desired. As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. The flavoring agents preferably are taste enhancing agents and can include artificial sweeteners such as aspartame, saccharin, dipotassium glycyrrhizinate, and stevia. Flavoring agents may also include cinnamon oil, oil of wintergreen, peppermint oils, menthol, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particular flavors are the grape and cherry flavors and citrus flavors such as orange. Additional taste enhancing agents are described in U.S. Pat. No. 6,027,746 and are incorporated herein by reference. Surfactants include soaps, synthetic detergents, and wetting agents. Suitable surfactants include cationic surfactants, anionic surfactants, non-ionic surfactants, and amphoteric surfactants. Examples of surfactants include Polysorbate 80; sorbitan monooleate; sodium lauryl sulfate (sodium dodecylsulfate); soaps such as fatty acid alkali metal salts, ammonium salts, and triethanolamine salts; cationic detergents such as dimethyl dialkyl ammonium halides, alkyl pyridinium halides, and alkylamine acetates; anionic detergents such as alkyl sulfonates, aryl sulfonates, olefin sulfonates, alkyl sulfates, olefin sulfates, ether sulfates, and monoglyceride sulfates, and alkyl sulfosuccinates, olefin sulfosuccinates, ether sulfosuccinates, monoglyceride sulfosuccinates, alkyl phosphates, alkyl phosphonates, potassium laureate, sodium lauryl sulfate, sodium dodecylsulfate, alkyl polyoxyethylene sulfates, dioctyl sodium sulfosuccinate, phosphatidyl glycerol, phosphatidylinositol, diphosphatidylglycerol, phosphatidyl inosine, phosphatidylserine, phosphatidic acid and their salts, cholic acid and other bile acids (e.g., cholic acid, deoxycholic acid, glycocholic acid, taurocholic acid, glycodeoxycholic acid) and salts thereof (e.g., sodium deoxychelate, etc.), and lecithin.; nonionic detergents such as fatty amine oxides, fatty acid alkanolamides, and poly(oxyethylene)-block-poly(oxypropylene) copolymers; and amphoteric detergents, for example, lecithin, alkyl β-aminopropionates and 2-alkylimidazoline quaternary ammonium salts; wetting agents such as, glycerin, proteins, and peptides; water miscible solvents such as glycols; and mixtures thereof. Enhancers are selected from the groups consisting of solubility enhancers, dissolution enhancers, permeability enhancers, stabilizers, enzyme inhibitors, p-glycoprotein inhibitors, multidrug resistance protein inhibitors and combinations thereof Solubilizers include cyclodextrins, vitamin E TPGS, oleic acid, menthol, sodium docusate, PEG-32 glyceryl plamitostearate, benzyl alcohol, povidone, combinations thereof, and others known to those of ordinary skill in the art. Preservatives include compounds used to prevent the growth of microorganisms. Suitable preservatives include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and others known to those of ordinary skill in the art. Examples of absorbents include sodium starch glycolate (Explotab™, Primojel™); croscarmellose sodium (Ac-Di-Sol®); polyvinylpyrrolidone (PVP) (e.g., Polyplasdone™XL 10); veegum; clays; alginates; alginic acid; carboxymethylcellulose calcium; microcrystalline cellulose (e.g., Avicel™); polacrillin potassium (e.g., Amberlite™); sodium alginate; corn starch; potato starch; pregelatinized starch; modified starch; cellulosic agents; montmorrilonite clays (e.g., bentonite); gums; agar: locust bean gum; gum karaya; pecitin; tragacanth; carragenans and other absorbents known in to those of ordinary skill in the art. Exemplary pore formers include water soluble polymers such as polyethylene glycols, lactose, sucrose, glucose, propylene glycols, and povidone; lactose, sucrose, glucose, salts such as calcium sulfate, calcium phosphate, sodium chloride, magnesium chloride and the like. Exemplary osmagents or osmotic agents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art. As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art. As used herein, bioadhesive and mucoadhesive agents include polymers, either water soluble or water insoluble, with or without crosslinking agents, known in the literature as being bioadhesive. Examples of bioadhesives include, but are not limited to: natural materials such as gums (e.g., karaya gum), carmelose, chitin, chitosan, carrageenans, eucheuma, fucoidan, hypnea, laminaran, furcellaran, agar, agarose, algin, amylose, scleroglucan, arabinoglactins, galactomannan, starches, alginates such as potassium and sodium, pectins, polypeptides such as gelatins, collagen and the like; cellulose materials including substituted and unsubstituted celluloses such as cellulose, ethycellulose, methylcellulose, nitrocellulose, propylcellulose, hydroxypropylcellulose, hyciroxyethylcellulose, carboxymethylcellulose and hydroxypropylmethylcellulose, cellulose derivates, alkylcellulose and hydroxyalkylcellulose derivatives wherein the alkyl group is 1 to 7 carbons, cellulose acetate butyrate and carboxyalkylceilulose; carboxyvinyi copolymers; polyethylene glycols; polyethylene oxides; polyethylene glycol ethers of aliphatic alcohols (such as cetyl, lauryl, oleyl and stearyl), polyhydroxyalkyl methacrylates, propylene glycol alginates; polyacrylamides; polyacrylic acids; vinyl polymers such as polyvinyl alcohol, polyvinyl ethers, polyvinyl acetate and polyvinylpyrrolidones; copolymers of hydroxyalkyl esters of acrylic and methacrylamide, N-vinyl-2-pyrrolidone, alkyl acrylates and methacrylates, vinyl acetate, acrylonitrile and styrene; acrylic acid-based polymers (e.g., carbomers such as the Carbopol® polymers); monoalkyl esters of poly (methyl vinyl ether/maleic acid) (e.g., Gantriz® polymers); thiolated polymers (thiomers) including thiol group bearing chitosan or poly(acrylic acid) polymers; Corplex® polymers, sucralfate; gliadin; kollidon; cholestyramine; Spheromers™(I, II & III); and generally, any physiologically acceptable polymer showing bioadhesive properties. Poly(ethylene oxide)-poly(propylene oxide)-poly(ethylene oxide) triblock copolymers, known as poloxamers, are non-ionic surfactants that may be used as wetting, solubilizing and stabilizing agents in various oral dosage forms. Poloxamers are also capable of significantly increasing the dissolution rate of a low-solubility drug, and/or sustaining the concentration of dissolved drugs. This may result in higher dissolved drug concentration, or higher bioavailability, or both. The use of poloxamers in rapidly dissolving oral dosage forms may improve the concentration of dissolved drug in a aqueous environment for drugs that are poorly soluble in the aqueous environment. Poloxamers are triblock copolymers composed of poly(ethylene oxide) (PEO)-poly(propylene oxide) (PPO), poly(ethylene oxide) (PEO) in the configuration: HO (PEO)ₐ (PPO)_{b} (PEO)ₐ H poloxamers, also sold under the name Pluronics and Lutrol (BASF Corporation), are crystalline or semi-crystalline materials having molecular weight ranging from about 2,000 to about 20,000 daltons. The molecular weight of pharmaceutical grade poloxamers ranges from about 7,000 to 18,000 daltons. Examples of poloxamers include, but are not limited to: Pluronic® F-68 (Poloxamer 188), Pluronic® F87 (Poloxamer 237), Pluronic® F108 (Poloxamer 338), Pluronic® F127 (Poloxamer 407, Lutrol F127) and the like. Pluronic® is a registered tradename for BASF Corporation for block copolymers of ethylene oxide and propylene oxide represented by the chemical structure HO(C₂H₄O)ₐ(C₃H₆O)_{b}(C₂H₄O)ₐH wherein for: (a) Pluronic® F-68, a is 80 and b is 27; (b) Pluronic® F87, a is 64 and b is 37; (c) Pluronic® F108, a is 141 and b is 44; and Pluronic® F127, a is 101 and b is 56. The average molecular weights of these block copolymers are 8,400, 7,700, 14,600 and 12,600 for Pluronic® F-68, Pluronic® F-87, Pluronic® F108 and Pluronic® F127, respectively. Crystallization inhibitors include polymers that interact with the active agent to inihibit crystallization of the active agent during storage. Polymers that may be used as crystallization inhibators include, either alone or in combination, polyvinylpyrrolidone polymers, hydroxypropylmethylcellulose (HPMC, e.g., Methocel E5 Premium), HPMC phthalate, ethylcellulose, hydroxyethylcellulose, sodium carboxymethylcellulose (carmellose sodium), calcium carboxymethylcellulose, dextran, acacia, starches such as sodium starch glycolate (SSG, e.g., Explotab of Mendell), b-cyclodextrin (e.g., Kleptose of Roquette), block copolymers of ethylene oxide and propylene oxide (e.g., Pluronic F-68 and F-108), polyvinyl alcohol and polyethylene glycol (PEG). Forming compositions of an amorphous drug without stabilization can potentially lead to crystallization of the drug over time, leading to poor performance. Thus, there is a continuing need to provide methods and formulations that would provide amorphous/nanocrystalline drug that is physically stable and provide enhanced concentration of drug in aqueous solution as well as enhanced bioavailability. Solid compositions that include one or more crystallization inhibitors provide good physical stability. As used herein, “physically stable” or “physical stability” means the tendency of the amorphous or nanocrystalline drug present in the formulation to crystallize at ambient storage conditions of 25° C. and less than 20% RH. The physical stability of drug particles is greater due to the lower molecular mobility of the amorphous and nanocrystalline drug in the drug-containing phase (s) as well as the lower tendency of the amorphous drug to crystallize from the drug-rich phases. An improvement in physical stability may be determined by comparing the rate of crystallization of the drug in a “test composition” comprising a drug and poloxamer with the rate of crystallization of the drug in the control composition. The rate of crystallization may be measured by any standard physical measurements, such as x-ray diffraction, DSC, hot stage microscopy, solid state NMR or SEM analysis. Drug in a physically stabile condition will crystallize at a slower rate than the drug in the control composition. A relative degree of improvement in physical stability may be used to characterize the improvement in physical stability obtained by the composition. The “relative degree of improvement in physical stability” is defined as the ratio of the rate of crystallization in the control composition and the rate of drug crystallization in the test composition. For example, if the drug in the control composition crystallizes at a rate of 10% by weight per week and the drug in the test composition crystallizes at a rate of 5% by weight per week, the relative degree of improvement in physical stability would be 2. In some embodiments, compositions described herein provide a relative degree of improvement in physical stability of at least 1.5, at least about 2.0, or at least 3.0 relative to a control composition. It is believed that poloxamers (and other crystallization inhibitors) work to spatially confine the amorphous and nanocrystalline drug during the process. The formation of drug rich amorphous and nanocrystalline drug particles within the polymer matrix is believed to be one reason for the enhanced drug dissolution. The presence of poloxamer molecules in the matrix is also believed to improve the wetting properties because of the reduced surface tension as compared to the highly hydrophobic particles of the crystalline drug. As the drug/poloxamers particles are exposed to the dissolution medium or the GI fluid, due to its high hydrophilicity, the PEO chains of the poloxamers are first to interact with water to form hydrogen bonds. This allows fast penetration of water into the particles, which leads to fast disaggregation of the particles. As it proceeds, the amorphous/nanocrystalline drug particles are released with higher rates due to significantly increased surface area. In some embodiments, water-insoluble polymers may be included in the oral dosage form. Addition of water insoluble polymers may alter the dissolution and release rate of the estriol compound from the oral dosage form. Generally water insoluble polymers slow down and/or reduce the rate of release of the estriol compound from the oral dosage form. Examples of pharmaceutically-acceptable, water-insoluble polymers include, but are not limited to acrylic acid-based polymers, methacrylic acid based polymers, and acrylic acid—methacrylic acid based copolymers. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid. Derivatives of acrylic acid and methacrylic acid include, but are not limited to, alkyl ester derivatives, alkylether ester derivatives, amide derivatives, alkyl amine derivatives, anhydride derivatives, cyanoalkyl derivatives, and amino-acid derivatives. Examples of acrylic acid-based polymers, methacrylic acid based polymers, and acrylic acid—methacrylic acid based copolymers include, but are nor limited to to Eudragit® L100, Eudragit® L100-55, Eudragit® L 30 D-55, Eudragit® S100, Eudragit® 4135F, Eudragit® RS, acrylic acid and methacrylic acid copolymers, methyl methacrylate polymers, methyl methacrylate copolymers, polyethoxyethyl methacrylate, polycyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polyalkylmethacrylate, polyacrylamide, and polymethacrylic acid anhydride and glycidyl methacrylate copolymers. Further examples of pharmaceutically-acceptable, water-insoluble polymers include, but are not limited to, alkylcelluloses such as ethylcellulose, methylcellulose, calcium carboxymethyl cellulose, certain substituted cellulose polymers such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyvinyl acetate, polyester, waxes, shellac, zein, or the like. It should be understood that compounds used as excipients or that are used to modify the oral dosage form, may serve a variety of functions or purposes. Thus, whether a compound named herein is assigned to one or more classifications or functions, its purpose or function should not be considered as being limited to the named purpose or function. The pharmaceutically acceptable matrix material and excipients may be selected to produce an oral dosage form that rapidly dissolves or disintegrates when placed in the saliva of the buccal and/or sublingual cavity. As used in this application the term “rapidly disintegrating” means that the dosage formulation dissolves in an aqueous media within 5 minutes. In some embodiment, the oral dosage form dissolves within 4 minutes, within 3 minutes, within 2 minutes, within 1 minute or within 30 seconds. Rapid orally disintegrating oral dosage forms allow administration of a solid dosage form, for instance a tablet or film, that includes an estriol compound to a patient without the need to swallow the dosage form. The orally disintegrating form, due to its porous nature, disintegrates and, optionally dissolves, directly in the oral cavity, with the aid of saliva or, in some cases a small amount of water. The resulting released estriol compound is absorbed by the oral mucosal (buccal and sublingual) or the esophageal lining as it passes down to the stomach. Orally disintegrating tablets, contrary to candies, should disintegrate in a time to allow at least about 50%, at least about 60%, at least about 70%, at least about 80%, or at least about 90% of the estriol compound to be absorbed by the oral mucosa and/or the esophageal lining. For tablet formulations, porosity and disintegration times of the rapid orally disintegrating tablets may be determined using any known st.ndard technique. For example, tablet porosity (ε) may be calculated using the following equation: ε(%)=[1−M/ρV]×100 Where ρ is the true density and M (g) and V (cm³) are the weight and the volume of the tablet, respectively. Tablet volume is calculated from the diameter and thickness of the tablet measured with a micrometer. The true density of the powder is measured using a pycnometer (autopycnometer; 1320, Micromeritics, USA). The % porosity of tablets, in some embodiments, is between 10-40%; greater than 20%; or greater than 30% than that of regular tablets. The oral disintegration of tablets or films may be assessed in artificial saliva which has been correlated with in vivo disintegration behavior. One test methodology includes placing a tablet or film on a perforated grid with mesh openings of 0.8 mm immersed in a fixed volume (e.g., 18 mL) of artificial saliva maintained at 37±2° C. and recording the time taken for the tablet or film to disintegrate. Artificial saliva (pH 5.8) is composed of NaCl (0.4 g/L); KCl(0.4 g/L); CaCl₂.2H₂O (0.8 g/L); NaH₂PO₄2H₂O (0.78 g/L); NaS.9H2O (0.005 g/L); and urea (1 g/L). In some embodiments, the oral dosage form includes between about 0.1% to about 50%, between about 1% and about 30% by weight, between about 2% and about 20% by weight, or between about 5% and about 10% by weight of the polymeric material. In some embodiment, less than about 50%, less than about 40%, less than about 30%, less than about 20%, or less than about 10% of the oral dosage form by weight is the polymeric material. An estriol compound may be treated in a supercritical fluid to alter the morphology of the compound, as described above. In one embodiment, an estriol compound may be treated with a supercritical fluid to produce the estriol compound in an amorphous and/or nanocrystalline form. The amorphous or nanocrystalline estriol may be combined with one or more binders and one or more other excipients to form a tablet. In some embodiments, the tablet may release at least about 90% of the estriol compound in a time of less than about 300 seconds when contacted with saliva of the buccal cavity. The use of amorphous and/or nanocrystalline estriol may allow more rapid absorption of the drug through the mucosal surfaces of the oral cavity. Alternatively, an estriol compound may be treated using milling or recrystallization processes to produce an estriol compound in a micronized form. As used herein a “micronized form” is produced when a compound is composed of particles having an average particle size of less than about 50 μm. In one embodiment, the micronized estriol may be combined with one or more binders and one or more other excipients to form a tablet. In some embodiments, the tablet may release at least about 90% of the estriol compound in a time of less than about 300 seconds when contacted with saliva of the buccal cavity. The use of micronized estriol may allow more rapid absorption of the drug through the mucosal surfaces of the oral cavity. In another embodiment, amorphous and/or nanocrystalline estriol compound may be used to form a composition suitable for inhalation therapies (e.g., nasal administration). In one embodiment, the amorphous and/or nanocrystalline estriol compound may be combined with one or more inert carriers to form a composition suitable for inhalation. In other embodiments, micronized estriol compound may be combined with one or more inert carriers to form a composition suitable for inhalation therapies. The inert carrier may be any pharmacologically inert material or combination of materials which is acceptable for inhalation. The carrier particles, in some embodiments, are water-soluble particles, including, but not limited to, one or more of sugars/carbohydrates (e.g., crystalline sugars, monosaccharides, disaccharides, sucrose, lactose, lactose monohydrate, glucose, glucose monohydrate, maltose, arabinose, saccharose, trehalose), oligo- and polysaccharides (e.g., dextrane), polyalcohols (e.g., sorbitol, mannitol, or xylitol), salts (e.g., salts of potassium, calcium, magnesium, sodium chloride or calcium carbonate) alpha hydroxyacids (e.g., citric acid, tartaric acid), polymers (e.g., polyvinyl alcohol, polyethylene glycol, hydrophilic celluloses, HPC, HPMC), peptides, amino acids, proteins and macromolecules, surfactants (e.g., sorbitan 20, sorbitan 80, and the like, in combination with a solid carrier), electrolytes (e.g., salts of potassium magnesium) and various non-electrolytes, or any combination thereof. The inhalation formulations may further include additive materials on the surfaces of the carrier particles to promote the release of the drug particles from the carrier particles upon release from the inhaler device or inhalation. The skilled artisan will understand that the size of the carrier particle must be suitably chosen for the form of inhalation delivery (e.g., pulmonary or nasal delivery). An inhalation composition may be formed by dissolving or dispersing the estriol compound in a solvent or solvent mixtures with one or more pharmaceutically acceptable matrix materials. The resulting feed solution or suspension may be dried by spray drying or by using a spray granulation with the inert carrier to form an inhalant composition. The term spray drying refers to processes which involve atomization of the feed solution or suspension into small droplets and rapidly removing the solvent from the mixture in a processor/chamber with a strong driving force for the evaporation of the solvent(s) (e.g., hot process air, vacuum or a combination of both). The term spray granulation refers to processes where the solution or suspension is sprayed onto a suitable chemically and/or physically inert carrier. In certain embodiments, an inhalation composition may be formed by dissolving or dispersing the estriol compound in a solvent or solvent mixtures with one or more pharmaceutically acceptable matrix materials. The resulting feed solution or suspension may be dried by coating it onto an inert carrier in a fluidized bed processor, pan coater or rotavapor-like processor. Coating of the inert carrier may be performed using a bottom spray mode, tangential spray mode, or a top spray mode. In another embodiment, amorphous and/or nanocrystalline estriol compound may be used to form a bioadhesive or oral disintegrating film. In one embodiment, the amorphous and/or nanocrystalline estriol compound may be combined with one or more pharmaceutically acceptable matrix compounds to form a film suitable for transmucosal delivery. An oral film form, as used herein, is an oral dosage form that has a thickness of less than about 5000 microns (5 mm). In other embodiments, micronized estriol compound may be combined with one or more pharmaceutically acceptable matrix materials to form a film suitable for transmucosal delivery. Films may be formed by hot melt-extrusion processes, compression techniques, or solvent evaporation processes. In another embodiment, amorphous and/or nanocrystalline estriol compound may be used to form a monolithic solid oral dosage form that is suitable for transmucosal delivery of the estriol compound. Such solid oral dosage forms may be composed of one or more bioadhesive polymers, or may include a coating that renders the oral dosage form bioadhesive. In one embodiment, the amorphous and/or nanocrystalline estriol compound may be combined with one or more pharmaceutically acceptable matrix compounds to form a bioadhesive monolithic solid oral dosage form. In other embodiments, micronized estriol compound may be combined with one or more pharmaceutically acceptable matrix materials to form a bioadhesive monolithic solid oral dosage form. Bioadhesive monolithic oral dosage forms may be formed by hot melt-extrusion processes, injection molding and compression molding. Alternatively, the bioadhesive monolithic solid dosage form may be a tablet (formed as described above) which has been coated with a bioadhesive polymer. An estriol composition may be formed by dispersing an estriol compound into one or more pharmaceutical acceptable matrix materials through various processes. For example, a solvent-based process, a fusion-melt process, a hybrid fusion-solvent process or other dispersion processes such as supercritical fluid processes can be used to prepare an estriol solid dispersion composition. The estriol compound used to form the estriol composition may be in a crystalline, nanocrystalline, amorphous, or micronized state. In one embodiment, a solvent-based process is used to form an estriol composition. A solvent-based process uses a solvent, such as water, non-organic solvents, and/or organic solvents, to dissolve and intimately disperse the estriol compound with one or more pharmaceutical acceptable matrix materials. No particular limitation is imposed on how to remove the solvent. Examples of the various ways to remove the solvent include, but are not limited to, evaporation under reduced pressure; atomizing the solution by means of a spray dryer; and applying the solution to inert particles (silica, silicon dioxide, lactose, microcrystalline cellulose, and/or anhydrous dibasic calcium phosphate) placed in an apparatus such as a fluid bed granulator or a rotary granulator, to thereby cause the solvent to be evaporated. If possible, water is used to dissolve the estriol compounds to prepare a solid dispersion, due to its availability and non-toxicity. Other suitable solvents may also be used, for example, alcohols and acetone, for use with water-insoluble matrix materials. Since estriol and many estriol derivatives are soluble in water, alcohols and acetone, any of these solvents, alone or in combination may be used to prepare a solid dispersion. In a fusion-melt process, the estriol compounds, and the one or more pharmaceutical acceptable matrix materials, are melted together at temperatures at or above the melting point of either the one or more pharmaceutical acceptable matrix materials and/or the estriol compound. In the fusion-melt process, the estriol compound and one or more pharmaceutical acceptable matrix materials can first be blended and melted in a suitable mixer. The molten mixture is then cooled rapidly to provide a congealed mass. Alternatively, the one or more pharmaceutical acceptable matrix materials can be melted into a molten state before mixing with the estriol compound into a homogeneous state. The melted mixture of the estriol compound and the one or more pharmaceutical acceptable polymers may be congealed by lowering the temperatures and then prepared into pharmaceutical dosage forms, such as a solid dosage form, e.g., powder and tablets. Alternatively, the cooled mixture can be subsequently milled to produce a powder form. The milled powdered form may further be blended with additional fillers, lubricant, and/or binders and compressed into tablets. In still another embodiment, a hybrid fusion-solvent process may be used to prepare an oral dosage form. For example, if there is thermal instability and immiscibility between the estriol compound and the one or more pharmaceutical acceptable polymers, the estriol compound may initially be dissolved in a small quantity of a solvent and added to a molten pharmaceutical acceptable polymer. The solvent is then evaporated to generate a product that is subsequently milled to produce a solid dosage form, such as a powder form, or compressed into tablets. In still another embodiment, a solid dispersion may be formed by mixing the estriol compound and the one or more pharmaceutical acceptable polymers in a supercritical fluid; removing the supercritical fluid to generate a solid dispersion that is subsequently milled to produce a solid dosage form, such as a powder form, or compressed into tablets. In any of the embodiments described herein for making an estriol solid dispersion (e.g., melt extrusion, a solvent-based process, a fusion-melt process, a hybrid fusion-solvent process, or a supercritical fluid process) one or more surfactants may also be present during formation of the estriol solid dispersion to allow rapid disintegration or dispersion, as well as to improve drug availability by altering the permeability of the oral mucosa. Suitable surfactants may be anionic, cationic, zwitterionic or nonionic surfactants. Surfactants may constitute about 1 to 20% (w/w) based on the total weight of the pharmaceutical composition. In any of the embodiments described herein for making an estriol solid dispersion (e.g., melt extrusion, a solvent-based process, a fusion-melt process, a hybrid fusion-solvent process, or a supercritical fluid process) one or more poloxamers may also be present during formation of the estriol solid dispersion to improve drug stability of the estriol solid dispersion. Suitable poloxamers include the Pluronics® polymers. Poloxamers may constitute about 1 to 20% (w/w) based on the total weight of the pharmaceutical composition. In some embodiments, the estriol solid dispersion composition may be formed using one or more bioadhesive polymers according to the techniques described above. The processes (e.g., a solvent-based process, a fusion-melt process, a hybrid fusion-solvent process or a supercritical fluid process) may be designed to produce a monolithic solid estriol dispersion composition suitable for transmucosal delivery. Such compositions may be suitable for adhering to a mucosal surface of the buccal cavity without causing undue discomfort to the user. An estriol composition suitable for transmucosal delivery may be produced in the from of a film or may be formed as a solid having a size suitable for such delivery. In some embodiments, an estriol solid dispersion composition may be produced in a particulate form (e.g., either directly or by a subsequent milling process of an solid). The particulate estriol dispersion composition may be combined with one or more binders and one or more other excipients to form a tablet. In some embodiments, the tablet may release at least about 90% of the estriol composition in a time of less than about 300 seconds when contacted with saliva of the buccal cavity. In another embodiment, an estriol solid dispersion composition may be produced in a particulate form. The particulate estriol dispersion composition may be used to form a composition suitable for inhalation therapies (e.g., nasal administration). In one embodiment, the particulate estriol dispersion composition may be combined with one or more inert carriers to form a composition suitable for inhalation. In another embodiment, an estrioF solid dispersion composition may be produced in a particulate form. The particulate estriol dispersion composition may be used to form a bioadhesive or oral disintegrating film. In one embodiment, the particulate estriol dispersion composition may be combined with one or more pharmaceutically acceptable matrix compounds to form a film suitable for transmucosal delivery. Films may be formed by hot melt-extrusion processes, compression techniques, or solvent evaporation.processes. Mixtures may be produced using any suitable means. Well-known mixing means known to those skilled in the art include dry mixing, dry granulation, wet granulation, melt granulation, high shear mixing, and low shear mixing. Granulation generally is the process wherein particles of powder are made to adhere to one another to form granules, typically in the size range of 0.2 to 4.0 mm. Granulation is desirable in pharmaceutical formulations because it produces relatively homogeneous mixing of different sized particles. Granulated mixtures are formed by use of a granulator. Granulators can be low shear, medium shear, or high shear. Shear is the amount of mechanical force of the granulator. A low-shear granulator uses very little mechanical force to combine powders and binding solution. The fluid-bed granulator, the most commonly used low-shear granulator, uses a high volume of air flow to elevate powders in a chamber while the solution/suspension that contains the active agent(s) is sprayed onto the enteric polymer particles to form a light bond. A fluid-bed granulator does not impart mechanical energy but instead relies on the powder characteristics and the binding solution to form the lightly held powders into granules. After the granulation process is completed, the resulting mixture may be dried to remove at least a portion of the solvent. A dry granulation process is used to form granules without using a liquid solution. Dry granulation may be conducted on a press using a slugging tooling or on a roller compactor commonly referred to as a chilsonator. Wet granulation involves forming granules using a granulating fluid or wetting agent that is subsequently removed by drying. Wet granulation may use water to form a granulated mixture. Melt granulation is a process in which powders are transformed into solid aggregates or agglomerates while being heated. It is similar to wet granulation except that a binder acts as a wetting agent only after it has melted. Melt extrusion is a process in which powders are transformed into solid aggregates or defined structures (e.g. granules, rods, beads) using a melt extruder. All of these and other methods of mixing pharmaceutical formulations are well-known in the art. Formation of particulate forms and mixtures of compounds may be performed using a variety of milling techniques. Techniques that may be used for formation of particulates or for mixing of the components include, but are not limited to, impact milling, attrition milling, knife milling, and direct-pressure milling. Impact milling occurs when a hard object that applies a blunt force across a wide area hits a particle to fracture it. This milling action may be produced by a rotating assembly that uses blunt or hammer-type blades. Another type of impact mill is a jet mill. A jet mill uses compressed gas to accelerate the particles, causing them to impact against each other in the process chamber. Impact mills can reduce both fine powders and large chunks of friable material down to average particle sizes of 50 μm with mechanical impact mills, and less than 10 μm with jet mills. Mechanical impact mill types include hammermills, pin mills, cage mills, universal mills, and turbo mills. In attrition milling, nondegradable grinding media continuously contacts the material, systematically grinding its edges down. This milling action is typically produced by a horizontal rotating vessel filled with grinding media and tends to create free-flowing, spherical particles. Attrition mills can reduce materials down to an average particle size of less than 1 μm. One type of attrition mill is the media mill (also called a ball mill). In knife milling, a sharp blade applies high, head-on shear force to a large particle, cutting it to a predetermined size to create smaller particles and minimize fines. This milling action is produced by a rotating assembly that uses sharp knives or blades to cut the particles. Knife mills can reduce 2-inch or larger chunks or slabs of material down to 250 to 1,200 μm. Mill types include knife cutters, dicing mills, and guillotine mills. Direct-pressure milling occurs when a particle is crushed or pinched between two hardened surfaces. Two rotating bars or one rotating bar and a stationary plate generally produce this milling action. Direct-pressure mills typically reduce friable materials down to 800 to 1,000 μm. Types include roll mills, cracking mills, and oscillator mills. In one embodiment, an estriol transmucosal medicament is a hot-melt extruded film having a thickness of less than 5000 microns (5 mm). The composition used to form the extruded film includes: about 30-95% wt. of one or more cellulose based matrix materials; about 1-25% wt. of an acrylic acid-based polymer (e.g., a carbomer or a polycarbophil polymer); about 1-60% wt. of poly(ethylene oxide); about 0.1-10% wt. of an organic acid; about 0.01-10% wt. of an antioxidant; and about 1-50% wt. of an estriol compound. In some embodiments, the estriol compound may be in amorphous and/or nanocrystalline form. In other embodiments the estriol compound may be in micronized form. Further details regarding the process for making such a film may be found in U.S. Pat. No. 6,375,963, which is incorporated herein by reference. In one embodiment, an estriol transmucosal medicament is a hot-melt extruded film having a thickness of less than 5000 microns (5 mm). The composition used to form the extruded film includes: about 30-95% wt. of one or more cellulose based matrix materials; about 1-25% wt. of an acrylic acid-based polymer (e.g., a carbomer or a polycarbophil polymer); about 1-60% wt. of poly(ethylene oxide); about 0.1-10% wt. of an organic acid; about 0.01-10% wt. of an antioxidant; and about 1-50% wt. of an estriol composition. Further details regarding the process for making such a film may be found in U.S. Pat. No. 6,375,963, which is incorporated herein by reference. In one embodiment, an estriol transmucosal medicament is a hot-melt extruded film having a thickness of less than 5000 microns (5 mm). The composition used to form the extruded film includes: about 30-95% wt. of one or more cellulose based matrix materials; about 1-25% wt. of an monoalkyl ester of a methyl vinyl ether/maleic acid copolymer (e.g., Gantriz® polymers); about 1-60% wt. of poly(ethylene oxide); about 0.1-10% wt. of an organic acid; about 0.01-10% wt. of an antioxidant; and about 1-50% wt. of an estriol compound. In some embodiments, the estriol compound may be in amorphous and/or nanocrystalline form. In other embodiments the estriol compound may be in micronized form. Further details regarding the process for making such a film may be found in U.S. Pat. No. 6,375,963, which is incorporated herein by reference. In one embodiment, an estriol transmucosal medicament is a hot-melt extruded film having a thickness of less than 5000 microns (5 mm). The composition used to form the extruded film includes: about 30-95% wt. of one or more cellulose based matrix materials; about 1-25% wt. of an monoalkyl ester of a methyl vinyl ether/maleic acid copolymer (e.g., Gantriz® polymers); about 1-60% wt. of poly(ethylene oxide); about 0.1-10% wt. of an organic acid; about 0.01-10% wt. of an antioxidant; and about 1-50% wt. of an estriol composition. Further details regarding the process for making such a film may be found in U.S. Pat. No. 6,375,963, which is incorporated herein by reference. In an embodiment, an estriol transmucosal medicament may include at least two layers. A first layer may include an estriol compound/composition and release at least about 90% of the estriol compound in a time of less than about 300 seconds when contacted with saliva of the buccal and/or sublingual cavity. The second layer also includes an estriol compound/composition, wherein the second layer releases between about 10% and 50% of the estriol compound after 30 minutes of contact with the saliva of the buccal and/or sublingual cavity, and at least about 90% of the estriol compound in a time of less than about 4 hours when contacted with saliva of the buccal and/or sublingual cavity. The first layer may include a combination of an estriol compound/composition and a pharmaceutically acceptable matrix material, as has been described herein. The second layer may include, in one embodiment, an estriol compound/composition and a pharmaceutically acceptable matrix material that is the same, or similar, to the material used to form the first layer. The second layer may further be coated with a material that delays release or dissolution of the second layer material. Alternatively, the second layer may be formed from a material that is different from the first material and is chosen to produce the desired release rate without the use of a coating material. In another embodiment, an estriol transmucosal medicament may include at least two layers. A first layer may include an estriol compound/composition and release at least about 90% of the estriol compound in a time of less than about 300 seconds when contacted with saliva of the buccal and/or sublingual cavity. The second layer may include an progestogen compound, wherein the second layer releases between about 10% and 50% of the progestogen compound after 30 minutes of contact with the saliva of the buccal and/or sublingual cavity, and at least about 90% of the progestogen compound in a time of less than about 4 hours when contacted with saliva of the buccal and/or sublingual cavity. The first layer may include a combination of an estriol compound/composition and a pharmaceutically acceptable matrix polymer, as has been described herein. The second layer may include, in one embodiment, a progestogen and a pharmaceutically acceptable polymer that is the same, or similar, to the material used to form the first layer. The second layer may further be coated with a material that delays release or dissolution of the second layer material. Alternatively, the second layer may be formed from a material that is different from the first material and is chosen to produce the desired release rate without the use of a coating material. In one embodiment, an estriol transmucosal medicament is a film having a thickness of less than 5000 microns (5 mm) The estriol transmucosal film is formed by applying a bioadhesive composition to a backing layer. The bioadhesive composition includes: about 5-30% wt. of one or more polyvinyl pyrrolidone (PVP) polymers; about 10-50% wt. of a bioadhesive polymer; and about 1-50% wt. of an estriol compound. The bioadhesive composition may optionally include one or more polyhydric alcohols in an amount of about 7-40% wt. Suitable materials that can be used, singularly, in combination, as laminates or as coextrusions, to form the backing layer are well known in the art and include films or sheets of polyethylene, polyester, polypropylene, polyurethane, polyolefin, polyvinyl alcohol, polyvinyl chloride, polyvinylidene, polyamide, vinyl acetate resins, Barex®, ethylene/vinyl acetate copolymers, ethylene/ethylacrylate copolymers, metal-vapor deposited films or sheets thereof, rubber sheets or films, expanded synthetic resin sheets or films, non-woven fabrics, fabrics, knitted fabrics, clothes, foils and papers. In some embodiments, the estriol compound may be in amorphous and/or nanocrystalline form. In other embodiments the estriol compound may be in micronized form. Further details regarding the process for making such a film may be found in U.S. Pat. No. 6,562,363, which is incorporated herein by reference. In one embodiment, an estriol transmucosal medicament is a film having a thickness of less than 5000 microns (5 mm) The estriol transmucosal film is formed by applying a bioadhesive composition to a backing layer. The bioadhesive composition includes: about 5-30% wt. of one or more polyvinyl pyrrolidone (PVP) polymers; about 10-50% wt. of a bioadhesive polymer; and about 1-50% wt. of an estriol composition. The bioadhesive composition may optionally include one or more polyhydric alcohols in an amount of about 7-40% wt. Further details regarding the process for making such a film may be found in U.S. Pat. No. 6,562,363, which is incorporated herein by reference. In one embodiment, an estriol transmucosal medicament is in the form of a bioadhesive tablet. The bioadhesive tablet includes: about 5-50% wt. of one or more cellulose based matrix materials; about 0.5-10% of a water-insoluble acrylic acid-based polymer (e.g., a polycarbophil polymer); about 1-75% of a water-soluble acrylic acid-based polymer (e.g., a carbomer); about 1-75% wt. of a filler and/or binder; and an effective amount of an estriol compound (e.g. about 0.1-50% wt.). Examples of fillers and/or binders include starch, lactose, silica and talc. Magnesium stearate may also be present as a tablet lubricant. In some embodiments, the estriol compound may be in amorphous and/or nanocrystalline form. In other embodiments the estriol compound may be in micronized form. Further details regarding the process for making such a tablet may be found in U.S. Pat. No. 6,248,358, which is incorporated herein by reference. In one embodiment, an estriol transmucosal medicament is in the form of a bioadhesive tablet. The bioadhesive tablet includes: about 5-50% wt. of one or more cellulose based matrix materials; about 0.5-10% of a water-insoluble acrylic acid-based polymer (e.g., a polycarbophil polymer); about 1-75% of a water-soluble acrylic acid-based polymer (e.g., a carbomer); about 1-75% wt. of a filler and/or binder; and about 0.1-50% of an estriol composition. Examples of fillers and/or binders include starch, lactose, silica and talc. Magnesium stearate may also be present as a tablet lubricant. Further details regarding the process for making such a tablet may be found in U.S. Pat. No. 6,248,358, which is incorporated herein by reference. In one embodiment, an estriol transmucosal medicament is in the form of a bioadhesive tablet. The bioadhesive tablet includes: about 5-50% wt. of one or more polyethylene oxide polymers; about 0.5-10% of an acrylic acid-based polymer (e.g., a carbomer); and an about 0.1-50% of an estriol compound. Magnesium stearate may also be present as a tablet lubricant. In some embodiments, the estriol compound may be in amorphous and/or nanocrystalline form. In other embodiments the estriol compound may be in micronized form. Further details regarding the process for making such a tablet may be found in U.S. Pat. No. 6,241,529, which is incorporated herein by reference. In one embodiment, an estriol transmucosal medicament is in the form of a bioadhesive tablet. The bioadhesive tablet includes: about 5-50% wt. of one or more polyethylene oxide polymers; about 0.5-10% of an acrylic acid-based polymer (e.g., a carbomer); and about 0.1-50% wt. of an estriol composition. Magnesium stearate may also be present as a tablet lubricant. Further details regarding the process for making such a tablet may be found in U.S. Pat. No. 6,241,529, which is incorporated herein by reference. In one embodiment, an estriol transmucosal medicament is a film having a thickness of less than 5000 microns (5 mm) The film includes a polymer-containing layer that serves as an estriol reservoir. This layer includes an estriol compound and is capable of liberating it upon action of saliva. The film includes about 10-95% wt. of the reservoir polymer and about 0.5-40% wt. of the estriol compound. The reservoir polymer includes, but is not limited to one or more of polyvinyl alcohols, polyvinyl pyrrolidones, polyvinyl acetates, polyethylene glycols, polyethylene oxide polymers, polyurethanes, polyacrylic acids, polyacrylates, polymethacrylates, poly(methyl vinyl ether-maleic acid anhydrides), starch, starch derivatives, natural gums, alginates, pectins and gelatine, pullulan, gel-forming proteins, chitosan, agar-agar, agarose, carrageenan, xanthan, tragacanth, dextrane and cellulose ethers such as ethyl cellulose, hydroxyethyl cellulose, propyl cellulose, carboxyl methyl cellulose, sodium carboxymethyl cellulose, hydroxypropyl cellulose, hydroxypropyl methyl cellulose, hydroxypropyl ethyl cellulose, cellulose acetate. Further details regarding the process for making such a film may be found in U.S. Patent Publication No. 2007/0243217, which is incorporated herein by reference. In one embodiment, an estriol transmucosal medicament is a film having a thickness of less than 5000 microns (5 mm). The film includes a polymer-containing layer that serves as an estriol reservoir. This layer includes an estriol composition and is capable of liberating it upon action of saliva. The film includes about 10-95% wt. of the reservoir polymer and about 0.5-40% wt. of the estriol composition. Further details regarding the process for making such a film may be found in U.S. Patent Publication No. 2007/0243217, which is incorporated herein by reference. In one embodiment, an estriol transmucosal medicament is in the form of a rapidly dissolving tablet. The rapidly dissolving tablet includes: about 1-15% wt. of one or more disintegrants; about 30-90% of a carbohydrate polyol (e.g., mannitol, xylitol, sorbitol, maltitol, etc.); and about 10-75% wt. of an estriol composition. An estriol composition may include an estriol compound dispersed in a cellulose based matrix material in⁻a particulate form. A tablet lubricant may also be present. Further details regarding the process for making such a tablet may be found in U.S. Pat. No. 7,067,149, which is incorporated herein by reference. In one embodiment, an estriol transmucosal medicament is in the form of a rapidly dissolving tablet. The rapidly dissolving tablet includes: about 1-5% wt. of one or more lubricants; and the remainder of the tablet including an estriol composition. An estriol composition may include an estriol compound dispersed in a pharmaceutically acceptable matrix material in a particulate form. One or more disintegrants may also be present. Further details regarding the process for making such a tablet may be found in U.S. Pat. No. 6,316,029, which is incorporated herein by reference. In one embodiment, an estriol transmucosal medicament is in the form of an effervescent tablet. The effervescent tablet includes: an acidifying agent, gas-releasing alkaline compound, a pharmaceutically acceptable matrix material, and an estriol compound. In some embodiments, the estriol compound may be in amorphous and/or nanocrystalline form. In other embodiments the estriol compound may be in micronized form. Further details regarding the process for making such a tablet may be found in U.S. Pat. No. 6,649,186, which is incorporated herein by reference. In one embodiment, an estriol transmucosal medicament is in the form of an effervescent tablet. The effervescent tablet includes: an acidifying agent, gas-releasing alkaline compound, a pharmaceutically acceptable matrix material, and an estriol composition. Further details regarding the process for making such a tablet may be found in U.S. Pat. No. 6,649,186, which is incorporated herein by reference. Any estriol transmucosal medicament described herein may include a bioadhesive and/or mucoadhesive agent as an outer coating that is intended to assist in retaining the preparation in the buccal, sublingual and esophageal mucosa over an extended duration to permit complete absorption of drug. In one embodiment, the oral dosage forms disclosed herein may be used to reduce or inhibit symptoms associated with perimenopause. The method of achieving such amelioration includes administering, to said subject, a oral dosage unit, prepared according to any of the embodiments described herein, that includes an estriol compound at least once a day. In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An oral dosage form comprising an estriol compound and a pharmaceutically acceptable matrix material, wherein the oral dosage form releases at least about 90% of the estriol compound in a time of less than about 300 seconds when contacted with saliva of the buccal and/or sublingual cavity.

2. The oral dosage form of claim 1, wherein the oral dosage form comprises about 0.01 mg to about 2 mg of the estriol compound.

3. The oral dosage form of claim 1, wherein at least a portion of the estriol compound is in a stabilized amorphous form.

4. The oral dosage form of claim 1, wherein at least a portion of the estriol compound is in a stabilized nanocrystalline form.

5. The oral dosage form of claim 1, wherein the pharmaceutically acceptable matrix material comprises a polyethylene glycol polymer.

6. The oral dosage form of claim 1, wherein the pharmaceutically acceptable matrix material comprises a cellulose based matrix material.

7. The oral dosage form of claim 1, wherein the pharmaceutically acceptable matrix material comprises a vinyl based matrix material.

8. The oral dosage form of claim 1, wherein the pharmaceutically acceptable matrix material comprises a polyvinyl pyrrolidone polymer.

9. The oral dosage form of claim 1, wherein the pharmaceutically acceptable matrix material comprises an acrylic acid-based polymer, a methacrylic acid-based polymer, an acrylic acid—methacrylic acid based copolymer, or a mixture thereof.

10. The oral dosage form of claim 1, further comprising a filler.

11. The oral dosage form of claim 1, further comprising a taste enhancing agent.

12. The oral dosage form of claim 1, further comprising a disintegrant.

13. The oral dosage form of claim 1, further comprising a stabilizer.

14. The oral dosage form of claim 1, further comprising one or more poloxamers.

15. The oral dosage form of claim 1, further comprising a mucoadhesive/bioadhesive polymer.

16. The oral dosage from of claim 1, wherein the oral dosage form is in the form of a tablet.

17. The oral dosage form of claim 1, wherein the oral dosage form has a thickness of less than 5000 microns.

18. The oral dosage form of claim 1, wherein the oral dosage form comprises:
a first layer, wherein the first layer comprises the estriol compound and the pharmaceutically acceptable matrix material, and wherein the first layer releases at least about 90% of the estriol compound in a time of less than about 300 seconds when contacted with saliva of the buccal and/or sublingual cavity; and
a second layer that includes an estriol compound and a pharmaceutically acceptable matrix material, wherein the second layer releases between about 10% and 50% of the estriol compound contained therein after 30 minutes of contact with the saliva of the buccal and/or sublingual cavity, and at least about 90% of the estriol compound in a time of less than about 4 hours when contacted with saliva of the buccal and/or sublingual cavity.

19. The oral dosage form of claim 1, wherein the oral dosage form comprises:
a first layer, wherein the first layer comprises the estriol compound and the pharmaceutically acceptable matrix material, and wherein the first layer releases at least about 90% of the estriol compound in a time of less than about 300 seconds when contacted with saliva of the buccal and/or sublingual cavity; and
a second layer that includes an estrogen compound and a pharmaceutically acceptable matrix material, wherein the second layer releases between about 10% and 50% of the estrogen compound contained therein after 30 minutes of contact with the saliva of the buccal and/or sublingual cavity, and at least about 90% of the estrogen compound in a time of less than about 4 hours when contacted with saliva of the buccal and/or sublingual cavity.

20. The oral dosage form of claim 1, wherein the oral dosage form comprises:
a first layer, wherein the first layer comprises the estriol compound and the pharmaceutically acceptable matrix material, and wherein the first layer releases at least about 90% of the estriol compound in a time of less than about 300 seconds when contacted with saliva of the buccal and/or sublingual cavity; and
a second layer that includes a progestogen compound and a pharmaceutically acceptable matrix material, wherein the second layer releases between about 10% and 50% of the progestogen compound contained therein after 30 minutes of contact with the saliva of the buccal and/or sublingual cavity, and at least about 90% of the progestogen compound in a time of less than about 4 hours when contacted with saliva of the buccal and/or sublingual cavity.

21. A method of treating perimenopausal conditions in a subject comprising orally administering to a subject an oral dosage form comprising an effective amount of an estriol compound, and a pharmaceutically acceptable matrix material, wherein the oral dosage form releases at least about 90% of the estriol in a time of less than about 300 seconds when contacted with saliva of the buccal and/or sublingual cavity of the subject.

22-84. (canceled)
